(19) United States
(12) Patent Application Publication
(10) Pub. No.: US 2005/0112614 A1
(43) Pub. Date: May 26, 2005

(54) Self-assembled circuits and circuit patterns

(75) Inventors: Matthew M. Cook, Pasadena, CA (US); Paul W. K. Rothemund, Pasadena, CA (US); Erik Winfree, Altadena, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: California Institute of Technology, Pasadena, CA (US)

(21) Appl. No.: 10/848,513

(22) Filed: May 17, 2004

Related U.S. Application Data
(60) Provisional application No. 60/470,636, filed on May 15, 2003.

(51) Int. Cl. H01L 21/00; C12Q 1/68

(52) U.S. Cl. 435/6; 438/1

(57) ABSTRACT

A self-assembly method for circuit patterns includes generating a set of tiles, each of the tiles corresponding to a segment of molecules, the set of tiles comprising a set of rule tiles and a set of boundary tiles, each tile having one or more binding regions; assigning a label from a set of labels to each binding region; self interacting, with attractive forces, one or more of the tiles with one or more other tiles among the set of tiles; associating using selective interaction of at least one boundary tile from the set of boundary tiles with at least one rules tile from the set of rules tiles based upon at least a label from the one boundary tile and at label from the one rules tiles; and bonding at least one binding region of the one boundary tile with at least one binding region of the one rules tile.

### CROSS-REFERENCES TO RELATED APPLICATIONS

This application claims priority to U.S. Provisional Patent Application No. 60/470,636, filed May 15, 2003, titled “Algorithmically Self-Assembled Circuits and Templates,” of Matthew M. Cook et al., and is hereby incorporated by reference herein in its entirety for all purposes.

### STATEMENT AS TO RIGHTS TO INVENTIONS MADE UNDER FEDERALLY SPONSORED RESEARCH OR DEVELOPMENT

Work described herein has been supported, in part by the National Human Genome Research Institute (Grant No. P50 HG02370, Alpha Project), National Science Foundation Career Grant No. 0093486, DARPA BIOCOMP Contract F30602-01-2-0561, and NASA NRA2-37143. The United States Government may therefore have certain rights in the invention.

### BACKGROUND OF THE INVENTION

The present invention relates to apparatus and techniques for the formation of patterns. More specifically the present invention relates to apparatus and techniques for the self-assembly of patterns for circuits using a set of building blocks that include DNA segments.

The manufacture of small devices has become a goal of technologists to increase computing power, to create new paradigms for computing, to develop new biotechnology, such as new drugs, as well as a plethora of goals. Small devices that might fulfill these goals might include nanotechnology devices, microtechnology devices as well as devices of other dimensions.

It has been contemplated that small devices might be manufactured using small-patterns that might be manufactured according to a variety of techniques. Small pattern might have dimensions in the nanometer range, the micrometer range or other ranges. Technologies that have been used to manufacture small patterns include microscopy techniques, such as atomic force microscope (AFM) techniques, wherein atoms and/or molecules are mechanically positioned on a substrate by the cantilevered arm of an AFM to form the small patterns. While AFM techniques might be of use to produce a variety of periodic and non-periodic patterns, the placement of individual atoms and/or molecules to build a small pattern tends to be relatively slow. Another technology that has been used to manufacture small patterns includes the use of chemical agents, wherein the assembled chemical agents form the small pattern. While chemical agents have been used to manufacture small patterns, traditional patterns formed by these techniques tend to be periodic, thus limiting their usefulness for the manufacture of small patterns that might be used for the further manufacture of devices that are non-periodic.

Patterns might be constructed from building blocks, wherein each building block in the pattern is unique. Creating a unique building block specifically for its position within a pattern is a tedious and time consuming process. Therefore, it is desired to manufacture building blocks that can be used to generate patterns, and these are not unique for the pattern position.

Accordingly, there is a need for the generation of patterns that may be periodic or non-periodic, that are created from a relatively small number of building blocks, and that might match the structures of devices to be manufactured using the patterns as templates.

### BRIEF SUMMARY OF THE INVENTION

The present invention provides an apparatus and technique for the formation of patterns. More specifically the present invention provides an apparatus and technique for the self-assembly of patterns for circuits using a set of building blocks that include DNA segments.

According to one embodiment of the invention, a self-assembly method is provided for self-assembling a circuit pattern. The method includes generating a set of tiles, each of the tiles corresponding to a segment of molecules, the set of tiles comprising a set of rule tiles and a set of boundary tiles, each of the tiles having one or more binding regions; assigning a label from a set of labels to each binding region of each tile; self interacting, with attractive forces, one or more of the tiles with one or more other tiles among the set of tiles; associating using selective interaction of at least one boundary tile from the set of boundary tiles with at least one rules tile from the set of rules tiles based upon at least a first label from the one boundary tile and at least a second label from the one rules tiles; and bonding at least one binding region of the one boundary tile with at least one binding region of the one rules tile to form a first portion of a circuit pattern. According to a specific embodiment, the first label and the second label are the same. According to another specific embodiment, the method further includes associating using selective interaction the one boundary tile with at least a second boundary tile of the set of boundary tiles based upon at least a third label from the one boundary tile and a fourth label from the second boundary tile; and bonding the one boundary tile with the second boundary tile to form a second portion of the circuit structure. According to another specific embodiment, the third label and the fourth label are the same. According to another specific embodiment, the method further includes associating using selective interaction the one rules tile with at least a second rules tile of the set of rules tiles based upon at least a fifth label from the one rules tile and a sixth label from the second rules tile; and bonding the one rules tiles with the second rules tile to form a third portion of the circuit structure. According to another specific embodiment, the fifth label and the sixth label are the same. According to another specific embodiment, the set of label are a set of binding rules. According to another specific embodiment, the tiles are passive tiles. According to another specific embodiment, the circuit pattern includes at least one of a circuit pattern for a demultiplexer, a memory, a pseudowavelet matrix, a Sierpinski triangle, a binary adder, and a Hadamard matrix. According to another specific embodiment, the molecular structures include at least one of DNA (deoxyribonucleic acid) segments, proteins, porphyrins, and polydimethylsiloxane molding. According to another specific embodiment, selective interaction automatically facilitates the attraction and binding of the tiles. According to another specific embodiment, the tiles have at least one of molecular dimensions, nanometer dimensions, and micrometer dimensions.

According to another embodiment, a method is provided for self-assembling molecular structures for circuit element structures. The method includes generating a set of tiles, each of the tiles corresponding to a segment of molecules, the set of tiles comprising a plurality of rule tiles and a plurality of boundary tiles, each of the tiles having one or more edges; assigning a respective binding rule to each edge of each tile, each of the rules associated with one or more strength numbers of a plurality of strength numbers; assigning one or more interaction rules to each of the rules tiles; self interacting, with attractive forces, one or more of the tiles with one or more other tiles among the set of tiles; associating using selective interaction of a first boundary tile with a second boundary tile from the plurality of boundary tiles based upon at least a first binding rule from the first boundary tile and at least a second binding rule from the second boundary tile, the first binding rule and the second binding rule being from the plurality of binding rules from the respective binding rules from each edge of each tile; bonding at least one edge of the first boundary tile with at least one edge of the second boundary tile to form a first portion of a circuit structure; associating using selective interaction at least one rules tile with at least one of the first boundary tile or second boundary tile based upon a binding rule and an interaction rule associated with the one rules tile; and bonding the one rules tiles with at least the first boundary tile or the second boundary tile to form a second portion of the circuit structure.

According to another embodiment, an assembly method includes generating a set of self-assembly tiles including rule tiles and boundary tiles configured to form a circuit pattern that includes a boundary portion and a rules portion; assigning a binding rule from a set of binding rules to each edge of the self-assembly tiles, wherein each binding rule has an assigned strength number; assigning a boundary-interaction rule from a set of boundary-interaction rules to at least one edge of each of the rule tiles; forming the a first portion of the boundary portion of the circuit pattern with the boundary tiles according to at least one of the binding rules; and forming a rules portion of the circuit pattern with the rule tiles according to the formed first portion of the boundary portion, at least one of the binding rules, and at least one of the boundary-interaction rules.

According to another specific embodiment, an assembly method is provided that includes the use of a tile model for self-assembly of a DNA pattern, such that the DNA pattern is a Hadamard-matrix-circuit pattern. The method includes generating a set of self-assembly tiles configured to generate the Hadamard-matrix-circuit pattern; assigning a binding rule from a set of binding rules to each edge of the self-assembly tiles, wherein each binding rule has an assigned numerical strength; assigning a boundary-interaction rule from a set of boundary-interaction rules to at least one edge of each of the rule tiles self-assembling a boundary with the boundary tiles according to at least one of the binding rules; and self-assembling a rules portion the Hadamard-matrix-circuit pattern with the rule tiles according to the formed boundary, at least one of the binding rules, and at least one of the boundary-interaction rules.

According to another specific embodiment, an assembly method is provided that includes the use of a tile model for self-assembly of a DNA pattern, such that the DNA pattern is a According to another embodiment, an assembly method is provided that includes the use of a tile model for self-assembly of a DNA pattern, such that the DNA pattern is a demultiplexer-circuit pattern. The method includes generating a set of self-assembly tiles configured to generate the demultiplexer-circuit pattern; assigning a binding rule from a set of binding rules to each edge of the self-assembly tiles, wherein each binding rule has an assigned numerical strength; assigning a boundary-interaction rule from a set of boundary-interaction rules to at least one edge of at least one of the rule tiles and the boundary tiles; self-assembling a boundary with the boundary tiles according to at least one of the binding rules; and self-assembling a rules portion the demultiplexer-circuit pattern with the rule tiles according to the formed boundary, at least one of the binding rules, and at least one of the boundary-interaction rules. According to a specific embodiment, each self-assembly tile of a first sub-set of the self-assembly tiles represents a binary 1 and is associated with an AND-logic gate, each self-assembly tile of a second sub-set of self-assembly tiles represents a binary 0 and is associated with a NAND-logic gate, the AND-logic gates and NAND-logic gates being referred to as the logic gates, and a number of the boundary tiles in a first column of the circuit pattern are associated with inputs to a second column of the demultiplexer-circuit pattern that includes a number of the logic gates.

According to another specific embodiment, an assembly method is provided that includes the use of a tile model for self-assembly of a DNA pattern, such that the DNA pattern is a pseudo-wavelet-circuit pattern. The method includes generating a set of self-assembly tiles configured to generate the pseudo-wavelet-circuit pattern; assigning a binding rule from a set of binding rules to each edge of the self-assembly tiles, wherein each binding rule has an assigned numerical strength; assigning a boundary-interaction rule from a set of boundary-interaction rules to at least one edge of at least one of the rule tiles and the boundary tiles; self-assembling a boundary with the boundary tiles according to at least one of the binding rules; and self-assembling a rules portion the pseudo-wavelet-circuit pattern with the rule tiles according to the formed boundary, at least one of the binding rules, and at least one of the boundary-interaction rules.

According to another embodiment, a self-assembly system is provided that is configured to self-assemble a demultiplexer-circuit pattern. The system includes a boundary tile configured as a seed tile and associated with a wire; at least four boundary tiles respectfully associated with NAND-logic gates; at least one boundary tile associated with a wire; at least four rule tiles respectively associated with AND-logic gates; and at least four rule tiles respectively associated with NAND-logic gates.

According to another embodiment, a self-assembly system is provided that is configured to self-assemble a pseudo-wavelet-matrix-circuit pattern. The method includes a boundary tile configured to form a corner tile of a boundary portion of the pattern; at least three additional boundary tiles; at least six rule tiles each assigned a binary-zero value; and at least two rule tiles each assigned a binary-one value, wherein at least five of the rule tiles are tagged and the remainder of the rule tiles are un-tagged, and wherein at least one of the rule tiles assigned the binary-zero value and tagged is configured to grow adjacent to at least one of the rule tiles assigned the binary-one value and is configured to propagate the tag. According to a specific embodiment, the rule tiles that are tagged are configured to propagate information and suppress the expression of the information in a growth direction of the pattern.

According to another embodiment, a self-assembly system is provided that is configured to self-assemble a Hadamard-matrix-circuit pattern. The system includes a set of hexagonal-self-assembly tiles, wherein each side of each tile is associated with a boundary-interaction rule of a set of boundary interaction rules, and the set of boundary interaction rules includes at least four rules, and wherein each side of each tile is associated with a binding strength of one or a binding strength of three.

Numerous benefits are achieved by way of the present invention over conventional techniques. For example, patterns, such as circuit patterns, may be formed from a relatively small number of self-assembly tiles. Accordingly, each tile position in a pattern may not be associated with a unique tile specifically made for the tile position. Moreover, using a relatively small number of self-assembly tiles of the present invention, relatively large patterns may be self-assembled. For example, using N number of self-assembly tiles, a pattern having size 2^{N} might be self-assembled. Accordingly, exponential compression may be achieved for the number of self-assembly tiles used to form a pattern based on the total number of tiles included in a pattern. Moreover, patterns might be self-assembled that are period or non-periodic. For example, non-periodic patterns might be self-assembled for circuit patterns, antenna patterns, fluidic-transport-network patterns, optoelectronic patterns, electro-optic patterns, quantum-circuit patterns, nano-electromechanical-switch patterns, micro-electromechanical-switch patterns, matrix-circuit patterns, mechanical-device patterns (e.g., mechanical-memory patterns) and numerous other patterns. Depending on the particular embodiment, one or more of these benefits may be achieved. These and other benefits will be described in more detail throughout the present specification and more particularly below.

Various additional object, features, and advantages of the present invention can be more fully appreciated with reference to the detailed description and accompanying drawings that follow.

### BRIEF DESCRIPTION OF THE DRAWINGS

FIGS. 1A, 1B, and 1C are simplified diagrams of a set of self-assembly tiles and a time ordered sequence of events of the self-assembly of a binary-counter pattern according to an embodiment of the present invention;

FIG. 2 is a simplified diagram of a set of self-assembly tiles and a demultiplexer that might be found formed from the self-assembly tiles according to an embodiment of the present invention;

FIG. 3 shows two self-assembled demultiplexers at right angles configured to address a memory according to another embodiment of the present invention;

FIGS. 4A, 4B, and 4C are simplified diagrams of the Sierpinski triangle and a set of tiles configured to self-assemble the Sierpinski triangle according to an embodiment of the present invention;

FIG. 5 shows a comparison view of self-similar binary matrices;

FIG. 6A is a simplified diagram of a tile set of that is configured to self-assemble the Sierpinski triangle from the upper right corner;

FIG. 6B is a simplified diagram of a tile set of that is configured to self-assemble the pseudowavelet-matrix-circuit pattern from the upper right corner;

FIG. 7 is a simplified diagram of two types of hexagonal tile that may be included in a set of hexagonal titles that is configured to self-assemble the pattern shown in FIG. 8C;

FIGS. 8A, 8B, 8C, and 8D show a time ordered sequence of events for the stages of growth of the self-assembly tiles shown in FIG. 7;

FIG. 9 shows the successive stages of the “Plus” fractal underlying the pattern of red and green tiles shown in FIGS. 8C and 8D;

FIG. 10A shows the green tiles for the pattern shown in FIG. 8D and that are arranged like the “Plus” fractal shown in FIG. 9;

FIG. 10B shows the red tiles are arranged the same way as the green tiles of FIG. 10A, except the fractal is rotated 45° and centered on an empty point;

FIG. 11A shows the red and green patterns together;

FIG. 11B shows the correspondence between the “Plus” fractal and the recursive “L triomino” tiling;

FIG. 12 shows three views of the self-assembled Hadamard matrix; and

FIG. 13 shows how the left two columns of red- and green-hexagonal tiles are subdivided into four hexagons.

### DETAILED DESCRIPTION OF THE INVENTION

The present invention provides an apparatus and technique for the formation of patterns. More specifically, the present invention provides an apparatus and technique for the self-assembly of patterns for circuits using a set of building blocks that include DNA segments.

Control of pattern formation for relatively small patterns is a goal of chemists, material scientists, nanotechnologists as well as others. Patterns of relatively small size might include patterns that are of nanometer dimension, micrometer dimension or other dimensions. Relatively small patterns might be used for the manufacture of correspondingly small devices that might match the patterns. For example, small circuit elements might be coupled to building blocks of a pattern and might be configured to self-assemble as the building blocks (referred to herein as self-assembly tiles or simply tiles) self-assemble to form the pattern. Other modules, such as chemical agents, mechanical devices, or the like might alternatively be coupled to tiles to self-assemble larger chemical agents, such as drugs, mechanical machine or the like.

Self-assembly as referred to herein includes the formation of a pattern from tiles that are configured to form the pattern, for example, by mixing the tiles together in a tile mixture in which the pattern is assembled based on features of the tiles. According to various embodiments of the present invention, tiles might include DNA (deoxyribonucleic acid), proteins, porphyrins, or polydimethylsiloxane molding or might be created by one or more photolithographic processes. Tile features that might direct the self-assembly of a pattern might include binding rules and boundary-interaction rules. The binding rules and boundary-interaction rules may control the tile edges of a tile that might form bonds with the tile edges of other tiles for self-assembly of a pattern. With respect to the various molecules that tiles might be constructed from, the tile's edges might include or might be models for the binding domains (or binding regions) of the molecules (e.g., DNA segments). The binding rules and boundary-interaction rules are sometimes referred to herein collectively or individually as labels. The binding rules might include numerical strengths for the binding of tile edges and may provide for the discrimination of edges that might bind. Interaction rules might include rules that further control the tile edges that might bind. The binding rules and boundary-interactions rules might include a variety of forces and their interactive properties that might be used to bind tiles. For example, the binding rules and the boundary-interaction rules might include chemical forces (e.g., van der Waals forces, covalent electron forces etc.), magnetic forces, electrostatic forces, electrodynamic forces, hydrogen bonding, capillary forces, entropic forces, quantum forces or other forces). For example, if the tiles are formed from segments of DNA the binding rules and boundary-interaction rules might be van der Waals forces and/or covalent electron forces. It should be understood that the forgoing listed forces are exemplary and are not limiting on the invention as recited in the claims.

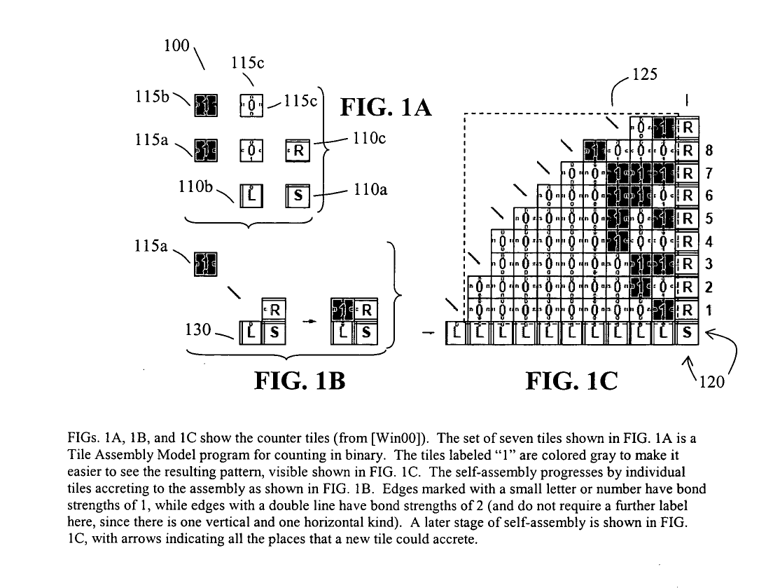

FIGS. 1A, 1B, and 1C are simplified diagrams of a set of self-assembly tiles 100 and a time ordered sequence of events of the self-assembly of a binary-counter pattern 105 according to an embodiment of the present invention. Self-assembly tiles include a set of boundary tiles 110 and a set of rules tiles 115. The embodiment is merely exemplary and should not unduly limit the scope of the claims herein. One of ordinary skill in the art would recognize many variations, alternatives, and modifications. Individual boundary tiles and rules tiles are labeled with the reference numbers 110 and 115, respectively, and an alphabetic suffix. The boundary tiles might be configured to self-assemble a boundary 120 of binary-counter pattern 105, wherein boundary 120 includes the right column of the pattern and the bottom row of the pattern. The boundary tiles include a seed tile 110a that is configured to form the corner of the boundary. The rules tiles are configured to bond to boundary 120 to form a rules portion 125 (outlined generally in phantom) of the demultiplexer-circuit pattern. According to some embodiment, a single seed tile forms the boundary portion of a pattern. According to some other embodiment, seed tiles might be dispersed in a pattern direction self-assembly at the boundary and in a rules portion 125 of the pattern. According to one embodiment, each self-assembled tile is assigned a boundary rule from a set of boundary rules that in part guide a self-assembly. The boundary rules might include numerical values for the strength bonds between tiles. For example, a bold bond line on the edge of a tile might indicate a strength-0 bond, a single narrow line might indicate a strength-1 bond, and two narrow lines might indicate a strength-2 bond. According to one embodiment, tiles might bond if the total binding strength is ≧2. For example, seed tile 110a might bond with boundary tile 110b using a single strength-2 bond, or a rules tile might bond to two boundary tiles or other rules tiles using two strength-1 bonds. According to alternate embodiments, tiles might be configured to bind if the total binding strength is ≧1 or ≧3. If the total binding strength is ≧1, self-assembly of a pattern may be less predictable than if the total binding strength for bonding is ≧2.

According to one embodiment, one or more edges of the self-assembly tiles are assigned a boundary-interaction rule from a set of boundary-interaction rules. The boundary-interaction rules are indicated by letters that are shown at the edges of the tiles. The edges of two tiles might bind if the boundary-interaction rules of the tiles match, and if the total binding strength is sufficient. For example, two tiles having the appropriate bonding strength might bond if the edges of two tiles each have the binding rule “c.” According to the embodiment shown in FIG. 1A, self-assembly tiles 100 include a total of 7 boundary-interaction rules having boundary-interaction rules 0, 1, c, and n. Further properties of the boundary-interaction rules are described in detail in the examples section below. Other embodiments might include more or fewer boundary-interaction rules, while still self-assembling the binary-counter pattern.

According to one embodiment, the self-assembly tiles are configured to self-assemble to form the binary-counter pattern by first forming a corner portion 130 of a boundary 120. Corner portion 130 includes the seed tile S, a lower boundary tile L, and a right boundary R. Note that the capital letters S, L, and R are placed on the boundary tiles for convenience to easily identify the locations of these tiles in the boundary. Other indicators might be placed on the boundary tiles to convey other information as is desired, or no indicators might be placed on the boundary tiles.

Subsequent to the self-assembly of corner portion 130, another boundary tile might grow on the corner portion, or a rules tiles 115a might bind to the L and R boundary tiles as shown in FIG. 1B. These steps might repeat to form the binary-counter pattern shown in FIG. 1C. And additional rules tiles may continue to self-assemble at the locations indicated by the diagonal arrow, and additional boundary tiles may continue self-assemble at the locations indicated by the vertical arrow and the horizontal arrow. As the tiles bind according to their binding rules and/or interactions rules (one or both sometimes being referred to as labels herein) the tiles are passive. That is the binding rules and boundary-interaction rules are not associated with mechanical means of binding the tiles together. Some tiles according to embodiments of the invention might include mechanical means to bind tiles or to supplement the binding rules and/or boundary-interaction rules to bind tiles. The mechanical means might include various latches, hooks or the like. Those of skill in the art will know of other mechanical means for binding tiles or for aiding the binding of tiles. Moreover, the binding rules and boundary-interaction rules self-assemble the tiles without additional (or external forces) coercing the self-assembly. The tiles may be mixed in solution that is configured to allow the tiles to interact, but does not provide forces the interfere with the binding of tiles.

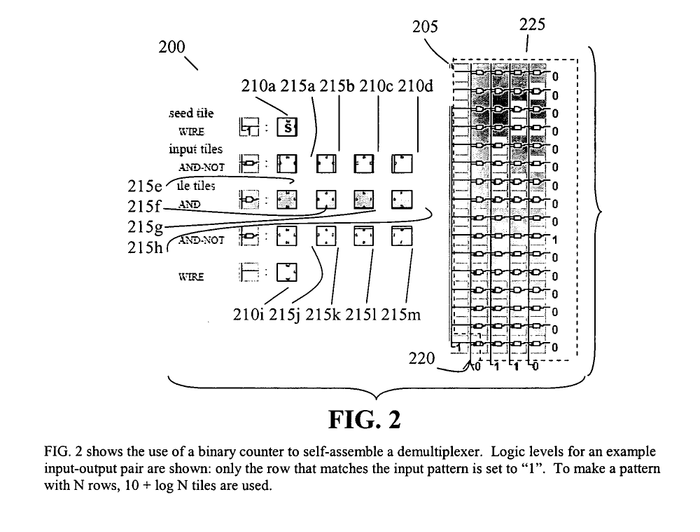

FIG. 2 is a simplified diagram of a set of self-assembly tiles 200 and a pattern 205 that might be self-assembled by self-assembly tiles 200 according to another embodiment of the present invention. The embodiment is merely exemplary and should not unduly limit the scope of the claims herein. One of ordinary skill in the art would recognize many variations, alternatives, and modifications. Pattern 200 may be a demultiplexer-circuit pattern, such that the bottom row of the pattern might be inputs (e.g., address bits) to the pattern and the rightmost column might be outputs of the pattern. As shown in FIG. 2, one output (having output 1) of the demultiplexer-circuit pattern is selected for activation. The self-assembly tiles might include a set of boundary tiles 210 and set of rules tiles 215. As referred to herein a set includes one or more elements. Each boundary tile and rules tile is labeled with the foregoing reference numbers and an alphabetic suffix. Similar to the binary-counter pattern described above, the boundary tiles might be configured to self-assemble a boundary 220 of the demultiplexer-circuit pattern, wherein the boundary 220 includes at least the seed tile 210a that is configured to form the corner of the pattern. The rules tiles are configured to grow on boundary 220 and grow on the rules tiles grow thereon, and form a rules portion 225 of the demultiplexer-circuit patterns. Rules portion 225 is indicated by the dashed line in FIG. 2. According to one embodiment, each tile is assigned a boundary rule from a set of boundary rules. The boundary rules might include numerical values for the strength bonds, such as strength-0 bond, a strength-1 bond, strength-2 bond, which are indicated by the line scheme described above. Tiles might bond if the total binding strength for each tile is ≧2.

According to one embodiment, one or more edges of the self-assembly tiles are assigned a boundary-interaction rule from a set of boundary-interaction rules. The edges of two tiles might bind if the boundary-interaction rules of the tiles match, and if the total binding strength is sufficient. According to the embodiment shown in FIG. 2, the self-assembly tiles include at total of 13 boundary-interaction rules. Other embodiments might include more or fewer boundary-interaction rules, while still self-assembling the demultiplexer-circuit pattern.

The self-assembly tiles might be associated with circuit elements that might form a demultiplexer circuit based on the demultiplexer-circuit pattern. For example, boundary tile 210a might be associated with a wire that is configured to provide inputs to the demultiplexer circuit. Various rules tiles might be associated with NAND-logic gates, AND-logic gates, and wires to form the rules portion 225 of the demultiplex circuit pattern.

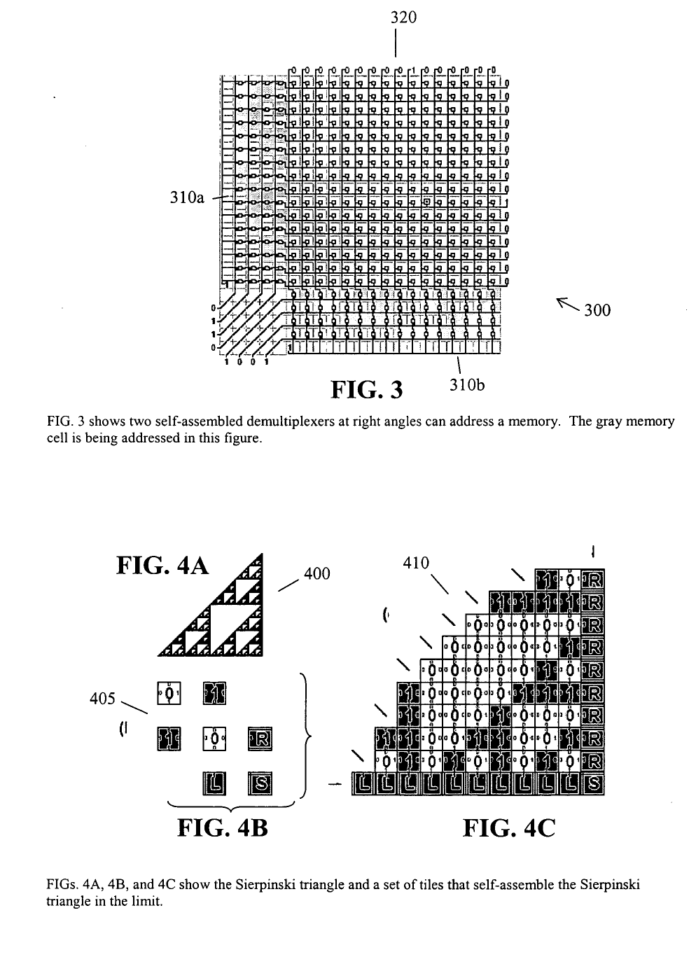

One or more self-assembly tiles might be added to boundary tiles 210 and/or rules tiles 215 to self-assemble other circuit patterns, such as the self-assembled memory-circuit pattern 300 shown in FIG. 3 that includes two demultiplexer-circuit patterns 310a and 310b coupled to the edges of a memory grid 320, wherein each memory element of the memory grid may be associated with a memory-circuit tile that is configured to self-assemble as the memory-circuit pattern self-assembles. It should be understood that the memory-circuit pattern is. one exemplary pattern that may be self-assembled by adding tiles to the set of tiles 200. Upon review of the instant description, claims, and attached drawings, those of skill in the art will be aware of a number of circuit patterns that might be self-assembled by adding and or altering the tiles in the set of tiles 200. Therefore, the embodiments described herein should not be viewed as limiting the scope of the invention, but should be viewed as illustrative embodiments of the invention as recited in the claims.

FIG. 4A is a diagram of a Sierpinski triangle 400, and FIG. 4B is a diagram of a set of self-assembly tiles 405 that are configured to self-assemble the Sierpinski triangle. FIG. 4C is a simplified diagram of a portion 410 of a Sierpinski triangle that might be self-assembled by the set of self-assembly tiles 405. The Sierpinski triangle might be self-assembled by the self-assembly tiles similarly to the binary-counter pattern and the demultiplexer-circuit pattern described above. It is noted that the relatively complex structures of the Sierpinski triangle may be assembled by as few as seven self-assembly tiles using as few as three boundary-interaction rules. Sierpinski triangles are known in the art for use as antennas, for example, for mobile telephones and the like, and for other circuit implementations. For example, self-assembly tiles 405 might be associated with conductor elements (e.g., nanometer wire segments, micrometer wire segments, carbon nanotubes or the like) and/or active circuit elements that self-assemble an antenna having the Sierpinski triangle pattern. Other features of the self-assembled Sierpinski triangle are described in detail in the examples section below.

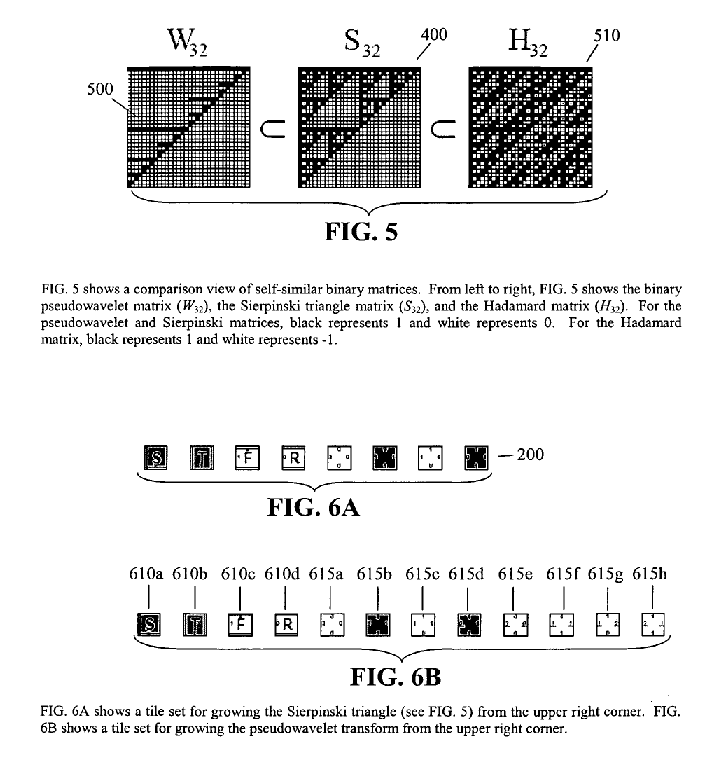

FIG. 5 is a simplified diagram of a set of self-assembled circuit patterns that might be self-assembled by various sets of self-assembly tiles according to embodiments of the present invention. Specifically, the right circuit pattern 500 shown in FIG. 5 is a binary-pseudowavelet-matrix-circuit pattern, the middle circuit pattern 410 is the Sierpinski triangle pattern, and the left circuit pattern is a Hadamard-matrix-circuit pattern 510. It is known in the art that the binary-pseudowavelet matrix and the Hadamard matrix are useful transform matrices, for example, for spectral analysis and may be used for Fast Fourier transformations or the like. FIGS. 6A and 6B provide a comparison view of sets of self-assembly tiles 200 and 605 configured, respectively, to self-assemble the Sierpinski triangle and the pseudowavelet-matrix-circuit pattern. The self-assembly tiles 605 include a set of boundary tiles 610 and a set of rules tiles 615 that are configured to self-assemble as described above. Rules tiles 615 include four “tagged” rules tiles 615e-615h that are not included in the set of self-assembly tiles 405. The tags are indicated by the underscored numbers zero and one at the side edges of tiles 615e-615h. The tagged rules tiles are configured to propagate information through the pseudowavelet-matrix-circuit pattern, but suppress the expression of the information. For example, the tags might be configured to propagate a preceding (according to a right to left self-assembly of the matrix) pattern of boundary-interaction rules that include a string of ones (wherein expressed information is all black, or all are tiles), and propagate this string of underscored ones. However, as tagged tiles 615e-615h are white tiles (or zero tiles), the information in the boundary-interaction rules that is propagated by these tiles is not expressed in the matrix, even though the information (string of ones boundary-interaction rules) is propagated. Using these tagged tiles, the black horizontal bars of the matrix might be grown and stopped as desired to form the pseudowavelet-matrix-circuit pattern. Other patterns, such as other circuit patterns, might be self-assembled using tagged tiles to stop or initiate the expression of information that might propagate through a self-assembly. Those of skill in the art will know of other patterns that might be self-assembled using tiles configured to suppress or initiate the expression of propagating information. Therefore, it should be understood the pseudowavelet-matrix-circuit pattern described above is one example of a pattern that might be self-assembled using the methods and apparatus of the present invention, and should not limit the invention as recited in the claims.

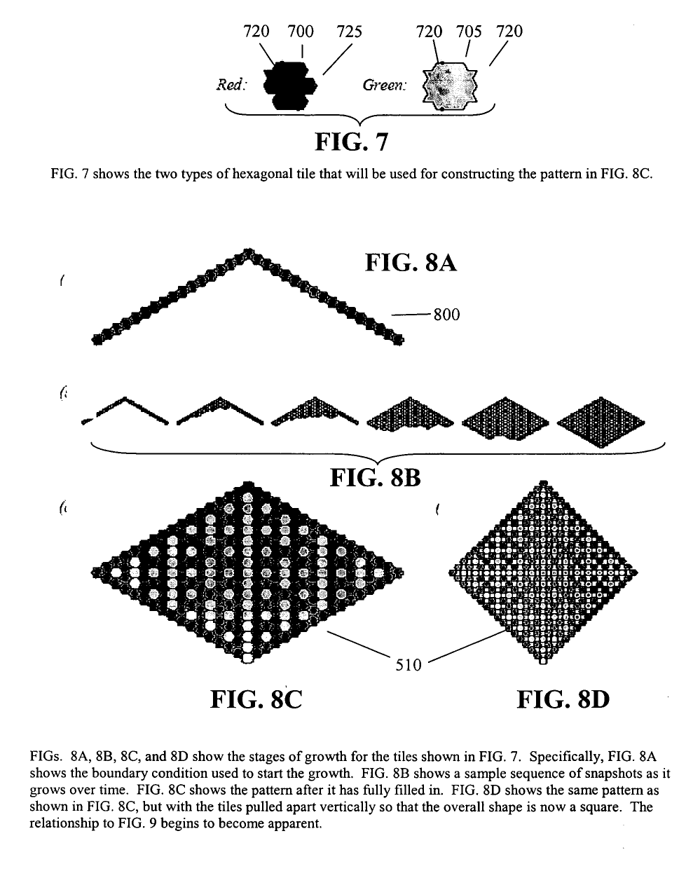

FIG. 7 shows two exemplary self-assembly tiles 700 and 705 that might be included in a set self-assembly tiles (not shown) that are configured to self-assemble the Hadamard-matrix-circuit pattern shown in FIG. 5. The set of self-assembly tiles might include twenty self-assembly tiles. Each of the twenty tiles of self-assembly tiles might be a hexagonal tile or a square tile. A set of hexagonal tiles might include four red-hexagonal tiles and sixteen green-hexagonal tiles. The descriptors “red” and “green” are used merely as a convenience to aid in the identification of the tiles. The tiles might just as well be referred to as blue and yellow tiles or orange and brown tiles or the like. The red-hexagonal tiles might include boundary tiles and rules tiles, and the green-hexagonal tiles might be rules tiles. The red-hexagonal tiles might also include a seed tile. The boundary tiles might be configured to self-assemble a boundary 800 as shown in FIG. 8A. FIG. 8B shows a time ordered sequence of events (time progresses from left to right) for the self-assembly of Hadamard-matrix-circuit pattern 510 shown in FIG. 8C. FIG. 8D shows the Hadamard-matrix-circuit pattern of FIG. 8C having the top and bottom edges stretched to form a square pattern.

According to one embodiment, select edges of the boundary tiles might be assigned strength-3 bonds, such that bonding of the boundary tiles might occur if the total strength bond of a tile is three. The edges of the rules tiles, which might include both red- and green-hexagonal tiles, might be assigned strength-1 bonds and might bond to other tiles if the total strength bond of a tile is three. Each edge of the red- and green-hexagonal tiles is assigned a boundary-interaction rule from a set of boundary interaction rules. The boundary-interaction rules are shown as triangular protrusions 720, triangular notch 725 and dots. As the dots might be positioned on a right side of an edge or a left side of an edge, the dots might represent two boundary-interaction rules depending on the right or left positioning. According to one embodiment, tiles may self-assemble if a protrusion is matched with a notch or if dots align.

Two or three of the top edges of the red- and green-hexagonal tiles might be configured as inputs to receive information from other tiles. Two or three of the bottom edges of the red- and green-hexagonal tiles may be configured as outputs, and may be configured output received information as a function of the received information. For example, a circuit having two or three inputs and two or three outputs might be associated with the red- and green-hexagonal tiles and might be configured to self-assemble as the Hadamard-matrix-circuit pattern self-assembles. As each tile might be associated with these numbers of inputs and outputs, relatively complex circuits might be constructed using the Hadamard-matrix-circuit pattern. For example, relatively complex logic circuits, such as FPGAs, PLDs, microprocessors or the like might be self-assembled with the self-assembly of the Hadamard-matrix-circuit pattern. Alternatively, a transformation circuit that implements the Hadamard matrix might be self-assembled. Those of skill in the art will know of other circuits and other devices that might be self-assembled with the self-assembly of the Hadamard-matrix-circuit pattern. Therefore, the foregoing described embodiments should not be understood as limiting of the invention, but should be understood to be illustrative examples.

A method for self-assembling molecular structures for circuit element patterns, according to an embodiment of the present invention may be outlined as follows:
1. generating a set of tiles, each of the tiles corresponding to a segment of molecules, the set of tiles comprising a set of rule tiles and a set of boundary tiles, each of the tiles having one or more binding regions;
1. assigning a label from a set of labels to each binding region of each tile;
2. interacting one or more of the tiles with one or more other tiles among the set of tiles;
3. associating using selective interaction of at least one boundary tile from the set of boundary tiles with at least one rules tile from the set of rules tiles based upon at least a first label from the one boundary tile and at least a second label from the one rules tiles; and
4. bonding at least one binding region of the one boundary tile with at least one binding region of the one rules tile to form a first portion of a circuit pattern.

Another embodiment of the present invention may be outlined as follows:
1. generating a set of tiles, each of the tiles corresponding to a segment of molecules, the set of tiles comprising a plurality of rule tiles and a plurality of boundary tiles, each of the tiles having one or more edges;
2. assigning a respective binding rule to each edge of each tile, each of the rules associated with one or more strength numbers of a plurality of strength numbers;
3. assigning one or more interaction rules to each of the rules tiles;
4. interacting one or more of the tiles with one or more other tiles among the set of tiles;
5. associating using selective interaction of a first boundary tile with a second boundary tile from the plurality of boundary tiles based upon at least a first binding rule from the first boundary tile and at least a second binding rule from the second boundary tile, the first binding rule and the second binding rule being from the plurality of binding rules from the respective binding rules from each edge of each tile;
6. bonding at least one edge of the first boundary tile with at least one edge of the second boundary tile to form a first portion of a circuit structure;
7. associating using selective interaction at least one rules tile with at least one of the first boundary tile or second boundary tile based upon a binding rule and an interaction rule associated with the one rules tile; and
8. bonding the one rules tiles with at least the first boundary tile or the second boundary tile to form a second portion of the circuit structure.

The above sequences of steps provide methods according to embodiments of the present invention. Alternatives can also be provided wherein steps are added, one or more steps are removed, or one or more steps are provided in a different sequence without departing from the scope of the claims herein. Further details of these methods can be found throughout the present specification.

Although the above has been described in terms of specific embodiments, there can be other variations, modifications, and alternatives. For example, while the patterns described above have been described as circuit pattern, the patterns might be useful for other self-assembly purposes. For example, the self-assembly tiles and patterns might be used to self-assemble chemical agents (e.g., to manufacture drugs), carbon nanotubes, carbon nanotube transistors, Buckey-balls, mechanical devices (to self-assemble e.g., nanometer-dimensional machines, micrometer-dimensional machines, such as mechanical memories or the like), fluidic devices (e.g., fluidic transport networks). Moreover, other pattern might be self-assembled such a fractal patterns or the like. These and other variations will be further described throughout the present specification and more particularly below.

### EXAMPLES

To illustrate certain principles and operations of the present invention, we have theoretically studied certain aspects of the invention defined herein and described aspect of this theoretical work below. As will be appreciated, this theoretical work is merely exemplary and should not unduly limit the scope of the claims herein. One of ordinary skill in the art would recognize many variations, modifications, and alternatives. Also, the theoretical examples described herein are merely intended to assist the reading in understanding certain aspects of the invention without limiting the claims as recited herein.

To expand on the same basic concepts introduced above, it is noted that self-assembly is a process in which basic units aggregate under attractive forces to form larger compound structures. Recent theoretical work has shown that pseudo-crystalline self-assembly can be algorithmic, in the sense that complex logic can be programmed into the growth process. This theoretical work builds on the theory of two-dimensional tilings, using rigid square tiles called Wang tiles for the basic units of self-assembly, and leads to Turing-universal models, such as the Tile Assembly Model. Using the Tile Assembly Model, we show how algorithmic self-assembly can be exploited for fabrication tasks, such as constructing the patterns that define certain digital circuits, including demultiplexers, RAM arrays, pseudowavelet transforms, and Hadamard transforms. Since DNA self-assembly appears to be promising for implementing the arbitrary Wang tiles needed for programming in the Tile Assembly Model, algorithmic self-assembly methods, such as those presented in this paper may eventually become a viable method of arranging molecular electronic components, such as carbon nanotubes, into molecular-scale circuits.

As an introduction to the notion of embedding computation in self-assembly, consider the simple example shown in FIGS. 1A, 1B, and 1B. The seven square tiles shown in FIG. 1A are Wang tiles, meaning that a pattern, such as that shown in FIG. 1C, is to be constructed by using as many copies of the tiles as desired, but the tiles may not be flipped or rotated, and the labels on the sides of abutting tiles match.

To be applicable to the subject of self-assembly, Wang's tiling model is extended to describe how the tiles aggregate into patterns, based on simple local rules. The Tile Assembly Model does this by assigning an integer-bond strength to each side of each tile. Growth occurs by the addition of single tiles, one at a time. In order for a new tile to attach itself to an existing pattern of tiles, the sum of the bond strengths on the edges where it would stick sum to at least the threshold τ, a fixed parameter of the experiment.

The tiles shown in FIG. 1A constitute a self-assembly program for counting in binary, and we will refer to them in this paper as the counter tiles. Lines on the edges are drawn to indicate the strength of binding: a thin line indicates a strength-1 bond, thin double lines indicate a strength-2 bond, and a thick line indicates a strength-0 bond (i.e. a side that does not stick to anything). Of course, a bond is formed if the edge labels (i.e., boundary-interaction rules) match.

To understand how the program works, we can conceptually categorize the seven tiles used in this example into two groups: The three tiles bearing large letters, called boundary tiles, are used to set up the initial conditions on the boundary of the computation. The four tiles bearing large numbers, called rule tiles, perform the computation and their numbers are to be interpreted as the binary digits of the output pattern.

The pattern shown in FIG. 1C includes a stage of self-assembly with τ=2, so tiles can bind to one another when the total binding strength is ≧2. For example, an “L” tile may bond on either side to another “L” tile or on its right side to an “S” tile, using a single strength-2 bond. The rule tiles, which can form strength-1 bonds, can bind to an assembly if two or more bonds cooperate to hold the tile in place, since τ=2. Thus, at first, the counter tiles that can assemble are boundary tiles, via strength-2 bonds. After the boundary tiles have begun to assemble into a V-shape, can rule tiles begin binding at corner sites as shown in FIG. 1B. The rule tile shown there can form two strength-1 bonds, and it is the tile that can stick there.

Successive additions of rule tiles and boundary tiles would result in a structure like that shown in FIG. 1C whose rows may be read, from bottom to top, as an enumeration of binary numbers. To understand how this works, inspect the rule tiles. Consider the bottom and right sides of each rule tile as inputs, and the left and top sides as outputs. A rule tile fitting into a corner “reads” two input bits by matching bonds; one bit it reads is the identity of the digit below it and the other is the carry bit from the tile to its right (if “c”, carry=1; if “n”, carry=0). The number on the rule tile and the bond that it outputs on its top reflect the result of adding, modulo 2, the two input bits; the bond it outputs to its left reflects the resulting carry bit. Rule tiles thus copy the digits below them, unless a carry is indicated from the right. Initially the “L” boundary tiles present all zeros to the rule tiles from below; this starts the counting at zero. The “R” boundary tiles present a new carry bit for each row of the counter from the right; this adds 1 to each successive row of the counter.

It is clear from FIG. 1C that multiple corner sites may be available for binding rule tiles at the same time; the order in which tiles are added at these sites is not specified. Despite the nondeterministic nature of assembly, it can be shown that the infinite structure that is formed by the counter tiles is unique. This is essentially because a unique rule tile binds at each corner site. For the counter tiles, this in turn is a consequence of our requirement that a rule tile may be added by the cooperative formation of at least two bonds at once, that is, τ=2 while the rule tile bond strengths is each 1.

To understand why we use τ=2, consider what would happen if τ=1. Since bond strengths are integers, any Tile Assembly Model program with τ=1 would not be able to require a tile to match the assembly on more than a single side, which makes information processing difficult at best, and if a unique output is used, τ=1 self-assembly appears not to be Turing-universal in two dimensions.

If τ=2 is more powerful than τ=1, then why don't we try even higher values? The two-fold answer is that (A) there does not seem to be much to gain, since most Tile Assembly Model programs already work well with τ=2, and (B) the experimental conditions allow a tile to be able to distinguish between a total bond strength of τ vs. a total bond strength of τ-1, so experimentally it is good to maximize the ratio between these, which means minimizing τ.

Is the τ=2 assumption reasonable for physical systems? Real crystal growth does seem to approximate τ=2 growth with strength-1 bonds. The phenomena of faceting and supersaturation are both consequences of the rarity of steps that violate τ=2. If a programmable experimental system well-modeled by τ=2 can be perfected, then two-dimensional self-assembly can be used to build a binary counter, and in fact, two-dimensional τ=2 self-assembly is universal. That is, any computer program may be translated into a set of tiles that when self-assembled, simulate the computer program. But the stubbornly practical may still ask: What is such an embedding of computation in self-assembly good for?

In principle, we could use self-assembly wherever we use a conventional computer. In practice we do not expect that computation by self-assembly will be able to compete with the speed of traditional computer architectures on explicitly computational problems. Instead, factors such as the physical nature of the output and the ability to run the same program many times at once in parallel motivate us to look for fabrication problems: particular patterns or sets of patterns that have potentially useful properties (e.g. as templates for electronic circuits), and which are amenable to self-assembly.

Naively we might wonder, “Can we self-assemble the circuit for a contemporary CPU?” Assuming that we can create tiles that act as circuit elements (it is noted that periodic electrical networks of functional LEDs have already been self-assembled on the millimeter scale) what we are really asking is “Can we self-assemble the layout pattern for a CPU?” The answer, in theory, is yes, and we may do so without using any complex computation.

Any particular pattern, no matter how complex, can be self-assembled by assigning a unique tile type, with a unique set of binding interactions with its neighbors, to each position in the pattern. The resulting program is as big as the pattern itself, with every tile in the program being used just once in the pattern. This type of self-assembly program (called unique addressing) is undesirable because it is not efficient—an efficient program would use a small number of tile types compared to the size of the pattern. Instead, unique addressing uses the greatest number of tile types possible to create a pattern. In physical implementations it appears that creating unique tile types and unique specific binding interactions is expensive and difficult, so with currently-envisioned techniques it seems that unique addressing is impractical except for very small patterns.

For a circuit to be well-suited to self-assembly, its structure should have a highly methodical pattern. The simplest such pattern would be a periodic arrangement of units, such as occurs in a random-access memory circuit, shown in the upper right region of FIG. 3. Indeed, using DNA self-assembly to create a molecular-scale memory was suggested in. The pattern generated by the counter tiles, described in detail above, 1 is a somewhat more interesting pattern, yet still methodical, which we can see is why it was easy to implement via self-assembly. Below, we describe more circuits with methodical structure.

Looking again at the counter tiles, we can think about what similar programs we might be able to construct. The counter tiles use a constant number of tile types to form a structure that grows indefinitely in two directions. If we wish to form a structure of a specific chosen size, we construct a set of tiles that not only count, but also stop when the count is complete. Such efficient self-assembly programs for growing finite shapes have been presented in. Here, we use an improved construction wherein, in each successive row, the rightmost “0” is replaced by a “1” and all bits to its right are zeroed. If there is no rightmost “0”, it stops. In this construction, shown in FIG. 2, a set of log N input tiles are used to define the width of the counter; the assembly grows into a rectangle of exactly size N×(1+log N). Thus, the counter can be used to make relatively narrow structures of a chosen length. By adding an additional constant number of tiles, we can self-assemble N×N squares. In these examples, wedding computation with self-assembly addresses what is to chemists a difficult synthetic (fabrication) problem—how to make polymer or crystalline structures of a well-defined size.

Perhaps surprisingly, the binary counter itself happens to yield the layout for a useful circuit. In FIG. 2, each tile type is shown labeled with a circuit element, such as a wiring arrangement, an AND gate, or an AND-NOT gate. Once assembled, the tiles form a circuit with 4 input lines along the bottom and 2⁴=16 output lines along the right. This is a demultiplexer: the address bits on the input lines specify exactly one output line to be active. A larger circuit with n=log N input lines and N=2ⁿ output lines can be self-assembled by changing the n input tiles. Note that multiple types of tiles can carry the same circuit element. This is a common phenomenon: all the markings on all the tiles comprise rather more information than just the pattern that we care to create; this excess information is necessary to specify how to grow the pattern correctly.

This is our first example of self-assembly being used to create a useful circuit. It is noted that our approach, in which the self-assembled patterns are used as templates for fabricating functional circuits out of other materials, can be contrasted to work that uses the self-assembly process itself to perform either a fixed or reconfigurable computation. Whether or not this could be practical depends upon how the tiles are implemented physically and how the circuit elements are attached to the tiles. Let us speculate on a few possible approaches, each of which involves considerable challenges. For example, if the tiles were made of DNA (e.g., the 2×12 nm molecules in) and the circuit elements were small molecular electronic devices covalently attached to the DNA, some chemical post-processing could be necessary to make functional connections between the circuit elements. On the other hand, if again DNA tiles were used but now the labels were single-stranded DNA protruding from the tiles, then in a post-processing step after assembly is complete, larger circuit elements (e.g., DNA-labeled carbon nanotubes) could be arranged by hybridization to the self-assembled pattern, thereby forming the desired circuit. Alternatively, the tiles could be micron- or millimeter-scale objects with embedded conventional electronic components, as in. Algorithmic self-assembly has been demonstrated at this scale as well.

A demultiplexer could be used as a building block for a larger self-assembled circuit: a pair of demultiplexers oriented at right angles along the borders of an N×N memory allows a memory element to be accessed using 2 log N lines. Thus, a memory circuit may be self-assembled (see FIG. 3). What other circuits might be possible? Our next constructions derive from the observation that the demultiplexer circuit implements a generalized inner product of a binary vector by a binary matrix, with the binary function EQUALS substituting for multiplication and AND substituting for addition in the definition of matrix multiplication. That is, the circuit takes an n-bit binary vector, “multiplies” it by an n×2ⁿ size binary “counting” matrix, and outputs a 2ⁿ long vector. Similarly, a circuit for an arbitrary binary matrix multiplication could be created by self-assembling a circuit decorated with logic gates as appropriate for the matrix of choice.

Another complex pattern that may be created by a simple self-assembling computation is the Sierpinski triangle, shown in FIG. 4A. Seven tiles, shown in FIG. 4B, are used to self-assemble the pattern shown in FIG. 4C and whose limit is this triangular fractal pattern. As with the counter tiles, its construction depends on τ=2 assembly. By labeling the sides of the tiles as “input” and “output”, individual tiles can be seen to encode the binary function XOR. Diagonals of the assembly, interpreted as zeros and ones, form rows of Pascal's triangle modulo 2. It can also be seen that diagonals of the assembly are instantaneous descriptions of a one-dimensional cellular automaton. Aside from its interpretation as a computation, this pattern is beginning to find some practical uses; rendered in metal the Sierpinski triangle appears to be a superior cellular phone antenna.

Does the Sierpinski triangle also have a circuit interpretation like the binary counter? Perhaps not, but it inspires thought: interpreted as a binary matrix the Sierpinski triangle has many periodic rows whose periods are related by a logarithmic scaling. This suggests that using the Sierpinski triangle as a matrix multiplier might effect some transform similar to a wavelet or Fourier transform. In fact, binary versions of the wavelet and Fourier transforms, namely the binary pseudowavelet trans-form and the Hadamard transform, have self-similar matrices closely related to the Sierpinski triangle. Both these transforms have been used in signal processing and image compression. The Hadamard matrix in particular has uses from quantum computation to cell phones, and can be used directly for implementing a parallel Walsh transform. Many theoretical and practical uses have been studied for Hadamard matrices of size 2ⁿ.

Given the similarity of these transforms to the Sierpinski triangle, it seems reasonable to expect that there should exist simple tile sets that self-assemble into circuit patterns for computing them. This turns out to be correct.

In FIG. 5 we give a visual comparison of these matrices to the Sierpinski matrix. Their formal similarity can be seen from their recursive definitions: W₁=T₁=H₁=S₁=[1], and for n a power of 2,

$$\begin{matrix}{W}_{2 n} = [ \begin{matrix}{T}_{n} & {W}_{n} \\ {W}_{n} & 0\end{matrix} ] , \\ {T}_{2 n} = [ \begin{matrix}{T}_{n} & {T}_{n} \\ 0 & 0\end{matrix} ] , \\ {S}_{2 n} = [ \begin{matrix}{S}_{n} & {S}_{n} \\ {S}_{n} & 0\end{matrix} ] , \\ {H}_{2 n} = [ \begin{matrix}{H}_{n} & {H}_{n} \\ {H}_{n} & - {H}_{n}\end{matrix} ]\end{matrix}$$

The pseudowavelet transform Wₙ has a simple self-similar structure for which it seems likely we can find a simple self-assembly program; in fact, a straightforward modification of the Sierpinski tiles will suffice. First, modify the tiles so growth occurs from the right to the left (in FIG. 5); then make a “tagged” version of each rule tile such that in each row, the first “0” to the left of a “1” gets tagged, and tags propagate leftward. The black cells are defined by untagged tiles. These tiles are shown in FIG. 6. Although these tiles build unbounded patterns, patterns of defined size can be created by replacing the 4 boundary tiles with a binary counter, as in FIG. 2 and FIG. 3.

In this section, we describe a set of hexagonal tiles that deterministically constructs self-similar Hadamard matrices of order 2ⁿ. We begin by describing a simple set of “red and green” tiles that constructs a nice but non-Hadamard self-similar pattern. Then, we describe a slight elaboration on those tiles that results in the generation of a Hadamard matrix and indicate how to turn the given construction into one that works with square tiles. Finally, we describe a proof for the correctness of our construction.

According to one embodiment of the present invention, FIG. 7 shows two hexagonal types of tiles, one red and one green. Unlike the square Wang tiles discussed in earlier sections, these tiles may be rotated and/or flipped over. Where two tiles abut, the notches on the sides of the hexagons fit together (one out and one in), or, where there are spots instead of notches, the spots of the tiles match. During growth, a new hexagon will need to fit in with three existing hexagons, so we will have τ=3. (Later we will show how r can be reduced to 2 by converting the hexagons into square tiles.)

The boundary condition used to initiate growth is composed of red tiles as shown in FIG. 8A. As in the constructed pattern shown in FIG. 1, three boundary tile types with strength-3 bonds can be used to construct this initial condition, or tiles analogous to those in FIG. 2 could be used to self-assemble a boundary of size exactly 2ⁿ.

As the assembly grows, as shown in FIG. 8B, the resulting pattern is unique, since at any location where we might try to add a tile, the two notches and the spot always restrict our options so that there is at most one way to add a tile. We can see this by examining just 3 cases: If both notches point up, then we add a green tile oriented according to the spot. If both notches point down, we add a red tile (upside down from the one shown in FIG. 7) oriented according to the spot. If one notch is down and another notch is up, then we use a red tile, but it will fit if the spot is on the side where the notch points up. Luckily, we can prove that the spot will indeed be on this side, as described in detail below.

Here we will make some observations about the pattern produced by the tiles, without worrying about proofs. Then below, we will prove that the grown pattern has the self-similar nature being described.

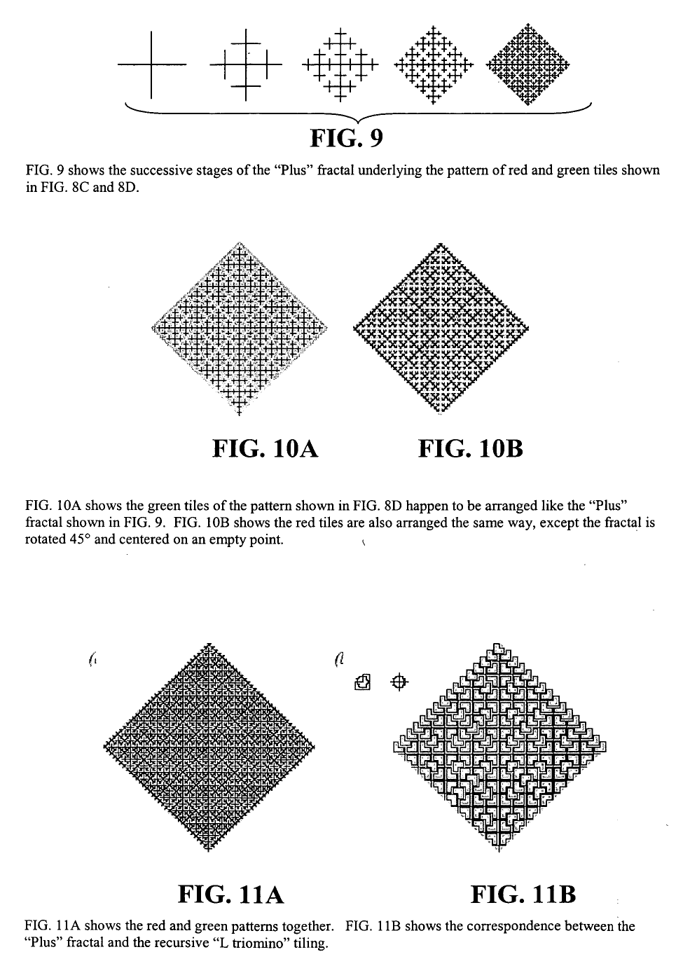

Given a fully grown pattern of size 2ⁿ+1, we can vertically stretch the rhomboidal array of hexagons shown in FIG. 8C into a square array as shown in FIG. 8D to make the self-similar pattern more apparent. The pattern is the self similar pattern of the “Plus” fractal shown in FIG. 9. As shown in FIG. 10, if we draw a green+on every green tile in the pattern, then we see exactly the “Plus” fractal, while if we draw a red X on every red tile, we see a simple rearrangement of the same fractal. In fact, we can draw both the red and the green pattern together on the same tiling, as shown in FIG. 11A, and in spite of them each covering the entire figure, the red and green fractals do not touch each other.

Since these “Plus” fractals have dimension 2, and adjacent tiles in the red pattern are \sqrt{2} times closer than adjacent tiles in the green pattern, there are twice as many red tiles as there are green tiles. On each red tile, instead of drawing a red X, we can draw an L triomino oriented according to the tile, yielding the well-known recursive tiling shown in FIG. 1B.

Now we will modify the red and green tiles to get a set of tiles that can generate a Hadamard matrix. The main modification is that we will add +1 and −1 markings to the tiles, so we will have a +1 red tile, a −1 red tile, a +1 green tile, and a −1 green tile. The +1 and −1 markings on these tiles are what will form the Hadamard matrix pattern.

We will have the red boundary tiles (corresponding to the pattern shown in FIG. 8A) all carry the +1 marking. The information about whether a tile is marked +1 or −1 will be propagated similarly to how the 0 and 1 markings were propagated in the tiles for the Sierpinski triangle, by labeling edges with “input” and “output” values. Specifically, on each tile we will label the two lower notched edges (the “output” edges) with the tile's main marking, while the two upper notched edges (the “input” edges) will be labeled with compatible inputs. Note that this means we can no longer rotate or flip our tiles, so we will need to explicitly have both orientations of the green tile, and all four orientations of the red tile.

On a red tile, the inputs may be either the same or different, and the tile's main marking always matches the input on the same side as the spot. On a green tile, the inputs are always the same, and the tile's main marking is always the opposite of the inputs. This results in a total of 16 types of red tile and 4 types of green tile.

Note that if we wanted to use square tiles instead of hexagonal tiles, we could eliminate the sides with the spots, and instead communicate the handedness of the spot via the tile to the left of what are now two square tiles touching at a corner. This breaks some of the symmetry of the tile set (although the marking could also be redundantly communicated on the right as well, to preserve symmetry), but if one needs to use τ=2 square tiles, it is nice to know that there is no theoretical obstacle.

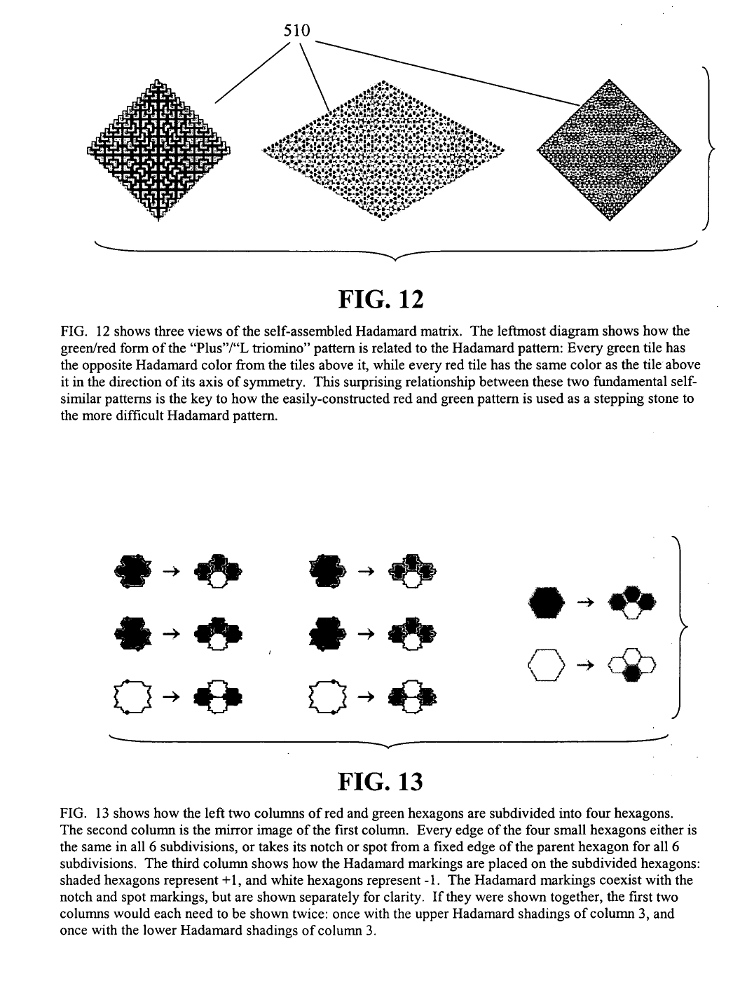

FIG. 12 shows the Hadamard pattern as it is produced on the hexagons together with the red and green patterns, on its own in the original hexagonal setting, and in its square matrix form. Now we know what to expect when we grow our pattern. Of course, to get a pattern of size exactly 2n for some given n, one would need to start with a boundary condition of just the right length, which can be accomplished as described in detail above.

In this section, we prove that the iterative process of tile accretion generates exactly the same pattern as the recursive subdivision process shown in FIG. 13. Since we know from the above description that at any given position there is one way a tile can be added to that location, we know that the pattern that grows is the unique pattern satisfying the boundary condition and the edge matching conditions on the tiles. (If there were more than one growable pattern, then the uppermost tile position differing in two such patterns would indicate a place where there is more than one way to add a tile.)

This means that if we can show that the recursive subdivision process always yields an arrangement that is consistent with the growth rules for the notch and spot markings on the edges as well as the Hadamard markings, then it yields exactly the same arrangement as is generated by the tile accretion process.

To show that the recursive subdivision process never leads to an inconsistency among the tiles, we consider what happens when we subdivide every tile in a consistent pattern X to get a more detailed pattern Y. We will show that if X was consistent, then so is Y.

First we consider the spots. The accretion rule for spots is that the spots line up on vertically adjacent tiles. Does the subdivision process guarantee that this will be the case throughout any pattern Y that is the result of subdividing a consistent pattern X? There are three cases where tiles in Y need to agree on the spot position: Two vertically adjacent tiles in Y may have come from (A) the same tile in X, (B) vertically adjacent tiles in X, or (C) diagonally adjacent tiles in X. For case (A), we know the spots will agree because we see that they agree in the interior of each individual subdivision rule. For case (B), we know the spots will agree because we see that the alignment of the spots at the top and bottom of each subdivision quadruple match the alignment of the spots at the top and bottom of the parent hexagon, and we know the parent hexagons agreed in X. For case (C), we know the spots will agree because both the lower and upper quadruple have the spot towards the top tile of the lower quadruple, regardless of what quadruples were used.

Next we consider the notches. The accretion rule for notches is that they match in direction on diagonally adjacent tiles. The subdivision process leads to three cases where two tiles in Y need to agree on the notch direction: (A) the two tiles are in the same quadruple, (B) the two tiles are the top tile of the lower quadruple and a side tile of the upper quadruple, or (C) the two tiles are a side tile of the lower quadruple and the bottom tile of the upper quadruple. For case (A), we know the notches will match because we can see that they match in every possible quadruple. For case (B), we know the notches will agree because regardless of which quadruples are involved, the notch in question will always match the original notch connecting the two parent tiles in X. For case (C), we know the notches will agree because regardless of which quadruples are involved, the notch will always point down.

Finally we consider the Hadamard markings. The accretion rule for the Hadamard markings is that for a red tile, the marking is copied from the upper left or upper right tile on the side of the spot, while for a green tile, the marking is the opposite of the markings on the upper left and upper right tiles (which happen to have the same markings). We can immediately see that the Hadamard markings obtained by subdivision obey the accretion rule for the side and bottom tiles of each quadruple, while for the top tile we need to know something about the upper left and upper right neighbor quadruples. What we know about these quadruples is that their side tiles have the same Hadamard marking as their parent in the X tiling. The top tile of the lower quadruple, whose Hadamard marking we are trying to verify, is also marked the same as its parent in the X tiling, and in fact it is always the very same tile as its parent. This means that the correct Hadamard marking for its parent in the X tiling is the same as its correct Hadamard marking in the Y tiling, and so since its parent was indeed marked correctly in the X tiling, we know it will be marked correctly in the Y tiling.

Since the spots, notches, and Hadamard markings present after subdivision follow all the rules used for accretion, we see that the subdivision process does indeed yield the growable patterns. If we start with the first tile shown in the subdivision rules, and repeatedly subdivide it to get patterns with more and more tiles, we see that the upper two sides of the resulting array of hexagons match exactly the boundary condition shown in FIG. 8. This means that the pattern obtained by repeated subdivision of this tile is exactly the same pattern that grows from that boundary condition. In particular, the Hadamard markings will be exactly those that occur on the pattern grown from the boundary condition. Since it follows from the definition of Hadamard matrices that they are exactly what gets produced by the Hadamard marking subdivision rule shown in the third column of FIG. 13, this means that the Hadamard markings on the grown pattern will exactly match the intended Hadamard matrix.

It is to be understood that the examples and embodiments described herein are for illustrative purposes only and that various modifications or changes in light thereof will be suggested to persons skilled in the art and are to be included within the spirit and purview of this application and scope of the appended claims. Each patent, patent application, and reference referred to herein by reference is hereby incorporated by reference herein in its entirety for all purposes.

1. A method for self-assembling molecular structures for circuit element patterns, the method comprising:
generating a set of tiles, each of the tiles corresponding to a segment of molecules, the set of tiles comprising a set of rule tiles and a set of boundary tiles, each of the tiles having one or more binding regions;
assigning a label from a set of labels to each binding region of each tile;
self interacting, with attractive forces, one or more of the tiles with one or more other tiles among the set of tiles;
associating using selective interaction of at least one boundary tile from the set of boundary tiles with at least one rules tile from the set of rules tiles based upon at least a first label from the one boundary tile and at least a second label from the one rules tiles; and
bonding at least one binding region of the one boundary tile with at least one binding region of the one rules tile to form a first portion of a circuit pattern.

2. The method of claim 1, wherein the first label and the second label are the same.

3. The method of claim 1, further comprising:
associating using selective interaction the one boundary tile with at least a second boundary tile of the set of boundary tiles based upon at least a third label from the one boundary tile and a fourth label from the second boundary tile; and
bonding the one boundary tile with the second boundary tile to form a second portion of the circuit structure.

4. The method of claim 3, wherein the third label and the fourth label are the same.

5. The method of claim 3, further comprising:
associating using selective interaction the one rules tile with at least a second rules tile of the set of rules tiles based upon at least a fifth label from the one rules tile and a sixth label from the second rules tile; and
bonding the one rules tiles with the second rules tile to form a third portion of the circuit structure.

6. The method of claim 22, wherein the fifth label and the sixth label are the same.

7. The method of claim 1, wherein the set of label are a set of binding rules.

8. The method of claim 1, wherein the tiles are passive tiles.

9. The method of claim 1, wherein the bonding steps occur without external forces effecting the formed bonds between the tiles.

10. The method of claim 1, wherein the bonding step includes bonding based on internal characteristics of the tiles.

11. The method of claim 1, wherein the circuit pattern includes at least one of a circuit pattern for a demultiplexer, a memory, a pseudowavelet matrix, a Sierpinski triangle, a binary adder, and a Hadamard matrix.

12. The method of claim 1, wherein the molecular structures include at least one of DNA (deoxyribonucleic acid) segments, proteins, porphyrins, and polydimethylsiloxane molding.

13. The method of claim 1, wherein selective interaction automatically facilitates the attraction and binding of the tiles.

14. The method of claim 1, wherein selective interaction includes cooperative binding of the tiles.

15. The method of claim 1, wherein the tiles have at least one of molecular dimensions, nanometer dimensions, and micrometer dimensions.

16. A method for self-assembling molecular structures for circuit element structures, the method comprising:
generating a set of tiles, each of the tiles corresponding to a segment of molecules, the set of tiles comprising a plurality of rule tiles and a plurality of boundary tiles, each of the tiles having one or more edges;
assigning a respective binding rule to each edge of each tile, each of the rules associated with one or more strength numbers of a plurality of strength numbers;
assigning one or more interaction rules to each of the rules tiles;
self interacting, with attractive forces, one or more of the tiles with one or more other tiles among the set of tiles;
associating using selective interaction of a first boundary tile with a second boundary tile from the plurality of boundary tiles based upon at least a first binding rule from the first boundary tile and at least a second binding rule from the second boundary tile, the first binding rule and the second binding rule being from the plurality of binding rules from the respective binding rules from each edge of each tile;
bonding at least one edge of the first boundary tile with at least one edge of the second boundary tile to form a first portion of a circuit structure;
associating using selective interaction at least one rules tile with at least one of the first boundary tile or second boundary tile based upon a binding rule and an interaction rule associated with the one rules tile; and
bonding the one rules tiles with at least the first boundary tile or the second boundary tile to form a second portion of the circuit structure.

17. The method of claim 16, wherein in selective interaction automatically facilitates the attraction and binding of the tiles.

18. The method of claim 16, wherein the tiles are passive devices.

19. The method of claim 16, wherein the binding rules are labels.

20. The method of claim 16, wherein the selective interaction of the first boundary tile and the second boundary tile automatically facilitates bonding the one edge of the first boundary tile with the one edge of the second boundary tile.

21. The method of claim 16, further comprising assigning one or more interaction rules to at least two of the boundary tiles

22. The method of claim 16, wherein the step of assigning one or more interaction rules to at least two of the boundary tiles includes assigning the one or more interaction rules to at least one edge of each of the at least two boundary tiles

23. The method of claim 16, further comprising assigning one or more interaction rules to at least one of the edges of each of the boundary tiles.

24. The method of claim 16, wherein the step of assigning one or more interaction rules to each of the rules tiles includes assigning one or more of the interaction rules to each edge of each of the rules tiles.

25. The method of claim 16, wherein the steps of associating and the steps of bonding include:
self-assembling the first boundary tile with the second boundary tile; and
self-assembling the at least one rules tile with at least one of the first boundary tile or second boundary tile.

26. The method of claim 16,
wherein the steps of self-assembling are based on cooperative binding of the tiles.

27. The method of claim 16, wherein the molecular structures includes at least one of DNA (deoxyribonucleic acid) segments, proteins, porphyrins, and polydimethylsiloxane molding.

28. The method of claim 16, wherein the circuit pattern is non-periodic.

29. The method of claim 16, where in the step of interacting including mixing the tiles, wherein the one or more of the tiles has the opportunity to interact one or more other tiles among the set of tiles.

30. The method of claim 16, wherein the circuit structure includes at least one of a circuit pattern for a demultiplexer, a memory, a pseudowavelet matrix, a Sierpinski triangle, a binary adder, and a Hadamard matrix.

31. The method of claim 16, wherein each of the tiles is one of a square or a hexagon.

32. An assembly method comprising:
generating a set of self-assembly tiles including rule tiles and boundary tiles configured to form a circuit pattern that includes a boundary portion and a rules portion;
assigning a binding rule from a set of binding rules to each edge of the self-assembly tiles, wherein each binding rule has an assigned strength number;
assigning a boundary-interaction rule from a set of boundary-interaction rules to at least one edge of each of the rule tiles;
forming the a first portion of the boundary portion of the circuit pattern with the boundary tiles according to at least one of the binding rules; and
forming a rules portion of the circuit pattern with the rule tiles according to the formed first portion of the boundary portion, at least one of the binding rules, and at least one of the boundary-interaction rules.

33. The method of claim 32, further comprising assigning a boundary-interaction rule from a set of boundary-interaction rules to at least one edge of at least two of the boundary tiles.

34. The method of claim 32, wherein the step of self-assembling the boundary portion with the boundary tiles includes self-assembling the boundary portion according to at least one of the binding rules and at least one of the boundary-interaction rules.

35. The method of claim 32, wherein the self-assembly tiles are configured to self-assemble a demultiplexer pattern that constitutes at least a portion of the circuit pattern.

36. The method of claim 35, wherein:
each self-assembly tile of a first sub-set of the self-assembly tiles represents a binary 1 and is associated with an AND-logic gate,
each self-assembly tile of a second sub-set of the self-assembly tiles represents a binary 0 and is associated with a NAND-logic gate, the AND-logic gates and NAND-logic gates being referred to as the logic gates, and
a number of the boundary tiles in a first column of the circuit pattern are associated with inputs to a second column of the demultiplexer-circuit pattern that includes a number of the logic gates.

37. The method of claim 32, wherein the set of self-assembly tiles is configured to self-assemble a memory-circuit pattern and at least one demultiplexer-circuit pattern, and these constitute portions of the circuit pattern.

38. The method of claim 32, wherein the steps of self-assembling the boundary portion and self-assembling the rules portion includes self-assembly by cooperative binding.

39. The method of claim 32, wherein the set of self-assembly tiles is configured to self-assemble a binary-pseudowavelet matrix that constitutes at least a portion of the circuit pattern.

40. The method of claim 39, wherein the rule tiles include a set of tagged tiles and set of un-tagged tiles.

41. The method of claim 40, wherein a self-assembly tile having a binary-zero value is grown adjacent to a self-assembly tile having a binary-one value and is a tagged tile having a tag that propagates in a growth direction.

42. The method of claim 32, wherein the set of self-assembly tiles is configured to self-assemble a Hadamard-matrix pattern that constitutes at least a portion of the circuit pattern.

43. The method of claim 42, wherein the self-assembly tiles are hexagonal tiles.

44. The method of claim 43, wherein the hexagonal tiles have at least two input edges and at least two output edges and the input edges are configured to receive information from tiles coupled to the input edges and the output edges are configured to propagate the received information or propagate a function of the received information.

45. The method of claim 44, wherein the input edges are associated with one-half of the hexagonal tiles and the output edges are associated with another half of the hexagonal tiles.

46. The method of claim 43, wherein the hexagonal tiles have three input edges and three output edges and the input edges are configured to receive information from tiles coupled to the input edges and the output edges are configured to propagate the received information or propagate a function of the received information.

47. The method of claim 42, further comprising assigning the number −1 to a first subset of the rule tiles and the number +1 to a second subset of the rule tiles.

48. The method of claim 42, wherein the set of binding rules includes numerical strengths of one and three.

49. The method of claim 32, wherein the set of self-assembly tiles includes tiles configured to propagate information and suppress the expression of the information in the circuit pattern.

50. The method of claim 32, wherein the circuit pattern includes a quantum-circuit pattern.

51. The method of claim 32, wherein the self-assembly tiles are formed from at least one of DNA (deoxyribonucleic acid), proteins, porphyrins, and polydimethylsiloxane molding.

52. The method of claim 32, wherein the self-assembly tiles are formed according to a photolithographic processes.

53. The method of claim 32, wherein the circuit pattern is non-periodic.

54. An assembly method that includes the use of a tile model for self-assembly of a DNA pattern, such that the DNA pattern is a Hadamard-matrix-circuit pattern, the method comprising:
generating a set of self-assembly tiles configured to generate the Hadamard-matrix-circuit pattern;
assigning a binding rule from a set of binding rules to each edge of the self-assembly tiles, wherein each binding rule has an assigned numerical strength;
assigning a boundary-interaction rule from a set of boundary-interaction rules to at least one edge of each of the rule tiles self-assembling a boundary with the boundary tiles according to at least one of the binding rules; and
self-assembling a rules portion the Hadamard-matrix-circuit pattern with the rule tiles according to the formed boundary, at least one of the binding rules, and at least one of the boundary-interaction rules.

55. The method of claim 54, further comprising assigning a boundary-interaction rule from a set of boundary-interaction rules to at least one edge of at least two of the boundary tiles.

56. The method of claim 54, further comprising modeling the self-assembly tiles as hexagonal tiles or as square tiles.

57. The method of claim 56, further comprising modeling the self-assembly tiles as hexagonal tiles.

58. The method of claim 57, wherein the hexagonal tiles have at least two input edges and at least two output edges and the input edges are configured to receive information from tiles coupled to the input edges and the output edges are configured to propagate the received information or propagate a function of the received information.

59. The method of claim 58, wherein the input edges are associated with a half of the hexagonal tiles and the output edges are associated with another half of the hexagonal tiles.

60. The method of claim 57, wherein the hexagonal tiles have three input edges and three output edges and the input edges are configured to receive information from tiles coupled to the input edges and the output edges are configured to propagate the received information or propagate a function of the received information.

61. The method of claim 57, wherein the set of binding rules includes numerical strengths of one and three.

62. The method of claim 54, wherein the binding rules and the boundary-interaction rules are cooperative-binding rules.

63. The method of claim 54, further comprising assigning the number −1 to a first subset of the rule tiles and the number +1 to a second subset of the rule tiles, wherein the values −1 and +1 are the values of the Hadamard-matrix-circuit pattern.

64. The method of claim 54, wherein the set of self-assembly tiles includes tiles configured to propagate information and suppress the expression of the information in the circuit pattern.

65. An assembly method that includes the use of a tile model for self-assembly of a DNA pattern, such that the DNA pattern is a demultiplexer-circuit pattern, the method comprising:
generating a set of self-assembly tiles configured to generate the demultiplexer-circuit pattern;
assigning a binding rule from a set of binding rules to each edge of the self-assembly tiles, wherein each binding rule has an assigned numerical strength;
assigning a boundary-interaction rule from a set of boundary-interaction rules to at least one edge of at least one of the rule tiles and the boundary tiles;
self-assembling a boundary with the boundary tiles according to at least one of the binding rules; and
self-assembling a rules portion the demultiplexer-circuit pattern with the rule tiles according to the formed boundary, at least one of the binding rules, and at least one of the boundary-interaction rules.

66. The method of claim 65, wherein:
each self-assembly tile of a first sub-set of the self-assembly tiles represents a binary 1 and is associated with an AND-logic gate,
each self-assembly tile of a second sub-set of self-assembly tiles represents a binary 0 and is associated with a NAND-logic gate, the AND-logic gates and NAND-logic gates being referred to as the logic gates, and
a number of the boundary tiles in a first column of the circuit pattern are associated with inputs to a second column of the demultiplexer-circuit pattern that includes a number of the logic gates.

67. The method of claim 65, wherein the binding rules and the boundary-interaction rules are cooperative-binding rules.

68. The method of claim 65, wherein the set of self-assembly tiles includes tiles configured to propagate information and suppress the expression of the information in the circuit pattern.

69. An assembly method that includes the use of a tile model for self-assembly of a DNA pattern, such that the DNA pattern is a pseudo-wavelet-circuit pattern, the method comprising:
generating a set of self-assembly tiles configured to generate the pseudo-wavelet-circuit pattern;
assigning a binding rule from a set of binding rules to each edge of the self-assembly tiles, wherein each binding rule has an assigned numerical strength;
assigning a boundary-interaction rule from a set of boundary-interaction rules to at least one edge of at least one of the rule tiles and the boundary tiles;
self-assembling a boundary with the boundary tiles according to at least one of the binding rules; and
self-assembling a rules portion the pseudo-wavelet-circuit pattern with the rule tiles according to the formed boundary, at least one of the binding rules, and at least one of the boundary-interaction rules.

70. The method of claim 69, wherein the set of self-assembly tiles are configured to self-assemble a binary-pseudowavelet matrix that constitutes at least a portion of the circuit pattern.

71. The method of claim 71, wherein the rule tiles include a set of tagged tiles and set of un-tagged tiles.

72. The method of claim 71, wherein a self-assembly tile having a binary-zero value is grown adjacent to a self-assembly tile having a binary-one value and is a tagged tile having a tag that propagates in a growth direction.

73. A self-assembly system configured to self-assemble a demultiplexer-circuit pattern comprising:
a boundary tile configured as a seed tile and associated with a wire;
at least four boundary tiles respectfully associated with NAND-logic gates;
at least one boundary tile associated with a wire;
at least four rule tiles respectively associated with AND-logic gates; and
at least four rule tiles respectively associated with NAND-logic gates.

74. The system of claim 73, further comprising a set of binding rules that includes at least three binding strengths.

75. The system of claim 73, further comprising a set of boundary-interaction rules that includes at least thirteen boundary-interaction rules.

76. The system of claim 75, wherein the boundary tiles include at least four of the boundary-interaction rules not included in the rule tiles.

77. A demultiplexer-circuit pattern formed from the boundary tiles and the rule tiles of claim 73.

78. A self-assembly system configured to self-assemble a pseudo-wavelet-matrix-circuit pattern comprising:
a boundary tile configured to form a corner tile of a boundary portion of the pattern;
at least three additional boundary tiles;
at least six rule tiles each assigned a binary-zero value; and
at least two rule tiles each assigned a binary-one value,
wherein at least five of the rule tiles are tagged and the remainder of the rule tiles are un-tagged, and wherein at least one of the rule tiles assigned the binary-zero value and tagged is configured to grow adjacent to at least one of the rule tiles assigned the binary-one value and is configured to propagate the tag.

79. The system of claim 78, wherein the rule tiles that are tagged are configured to propagate information and suppress the expression of the information in a growth direction of the pattern.

80. A pseudo-wavelet-matrix-circuit pattern formed from the boundary tiles and the rule tiles of claim 78.

81. A self-assembly system configured to self-assemble a Hadamard-matrix-circuit pattern comprising:
a set of hexagonal-self-assembly tiles, wherein each side of each tile is associated with a boundary-interaction rule of a set of boundary interaction rules, and the set of boundary interaction rules includes at least four rules, and wherein each side of each tile is associated with a binding strength of one or a binding strength of three.

82. The system of claim 81, wherein the set of tiles includes at least twenty types of tiles.

83. The system of claim 81, wherein the tiles have two input edges and two output edges and the input edges are configured to receive information from tiles coupled to the input edges and the output edges are configured to propagate the received information or propagate a function of the received information.

84. The system of claim 83, wherein the input edges are associated with a half of the tiles and the output edges are associated with another half of the tiles.

85. The system of claim 81, wherein the hexagonal tiles have three input edges and three output edges and the input edges are configured to receive information from tiles coupled to the input edges and the output edges are configured to propagate the received information or propagate a function of the received information.

86. A Hadamard-matrix-circuit pattern formed from the hexagonal tiles of claim 81.
